(19) United States
(12) Patent Application Publication
(10) Pub. No.: US 2012/0165670 A1
(43) Pub. Date: Jun. 28, 2012

(54) CONTRALATERAL ARRAY BASED CORRECTION OF TRANSCRANIAL ULTRASOUND ABERRATION

(75) Inventors: William Tao Shi, Briarcliff Manor, NY (US); Francois Guy Gerard Marie Vignon, Croton on Hudson, NY (US); Jeffry Earl Powers, Bainbridge Island, WA (US); Brent Stephen Robinson, Christchurch (NZ); Michael Richard Burcher, Impington (GB); Vijay Shamdasani, Seattle, WA (US)

(73) Assignee: KONINKLIJKE PHILIPS ELECTRONICS N.V., EINDHOVEN (NL)

(21) Appl. No.: 13/393,998

(22) PCT Filed: Aug. 25, 2010
(86) PCT No.: PCT/IB2010/053822

§ 371 Date: Mar. 2, 2012

Related U.S. Application Data
(60) Provisional application No. 61/239,455, filed on Sep. 3, 2009.

(51) Int. Cl. A61B 8/13

(52) U.S. Cl. 600/442

(57) ABSTRACT

Ultrasound aberration, especially in transcranial imaging or therapy, is corrected by capturing the laterally two-dimensional nature of the aberration in the ultrasound being received, as by means of a two-dimensional receiving transducer array (104, 108). In some embodiments, transmissive ultrasound (164) is applied through the temporal window and is, for example, emitted from one or more real or virtual point sources (160) at a time, each point source being a single transducer element or patch or the geometrical focus of a collection of elements or patches. A patch may serve, in one aspect as a small focused transducer in the near field. A contralateral array (104, 108) is, in one version, comprised of the point sources. In some aspects, aberration maps structured, independent-variable-wise, to correspond to the array structure of the receiving transducer embody aberration estimates, the ultrasound device being configured for improving ultrasound operation by modifying device settings to improve the location of ultrasound reception/transmission or correct beamforming. Enhancements include beam placement visualization, and intensity and beam shape prediction.

The present invention is directed to ultrasound aberration estimation and correction and, more particularly, to estimation by means of transmissive ultrasound.

Although stroke is one of the leading causes of death worldwide, acute stroke treatment is confined to thrombolytics such as tissue plasminogen activator (tPA).

Recent clinical studies have also shown that the addition of ultrasound to an accepted tPA therapy improves outcomes for ischemic stroke patients.

Because “time is brain” for stroke victims, it is desirable to make an early diagnosis and start some form of therapy as early as possible. There is clearly a need for a noninvasive and easily accessible method such as medical ultrasound to perform diagnosis, therapy, and treatment monitoring in emergency settings, such as in an ambulance.

The human skull has strong frequency-dependent aberration effects on ultrasound beams. Even the temporal bone (the thinnest part of the skull) can cause severe deflection, reflection, and attenuation of the ultrasound beam because of its convexity, surface roughness and the multiple impedances encountered by the ultrasound beam on the way into or back from the brain. These effects are highly variable from patient to patient and also strongly dependent on the location along the skull and orientation of ultrasound transducers, affecting both the efficacy and reproducibility of sonothrombolysis through the skull.

Adaptive aberration correction (refocusing) methods in the traditional pulse-echo mode have the potential to overcome these problems. Such methods, however, have so far gained little clinical acceptance for ultrasound imaging applications. They usually rely on noisy and poorly correlated signals backscattered by the tissues under investigation which yield poor estimates of the aberration, which is particularly problematic for transcranial ultrasound imaging with the strong insertion loss of the skull. Other experimental methods based on the computed-tomography-derived skull morphology are not practical in emergency settings because of limited availability of computed tomography (CT) to acute stroke patients and complex and time-consuming CT-ultrasound co-registration.

Experiments in a non-clinical setting have used a transducer as an ultrasound source to a receiving linear array, with the outside of a human skull bone adjacent to and facing the array and the ultrasound arriving incident to the inside of the skull bone. The arriving wavefront can, by adjusting aperture size of the transducer, be made regular, i.e., shaped like a section of a spherical surface, but becomes aberrated by the bone. Adjusting delays upon reception to restore regularity to the wavefront provides the basis for correcting ultrasound to be applied from the receiving side and through the bone. For clinical applications, the ultrasound to be applied in measuring aberration would have to pass through bones on both sides of the head, making attenuation a major problem. Lowering acoustic frequency was seen as a way to both increase signal-to-noise ratio (SNR) and decrease the impact of the aberration on the coherence of transcranial wavefronts. To compensate for the consequent increase in wavelength, an increased aperture was sought to restore resolution loss. However, the increased aperture size was found to make necessary some form of compensation or signal processing to approach diffraction limited resolution. See “Sampled Aperture Techniques Applied to B-Mode Echoencephalography,” by Phillips D. J., et al., Acoustic Holograms 6, 103-120 (1975).

The addition of contrast microbubbles in combination with ultrasound to tPA therapy has, according to recent clinical studies, been shown to improve outcomes for ischemic stroke patients.

However, currently the reproducibility and safety of microbubble-enhanced stroke therapy are severely compromised by uncertainties with regard to transcranial ultrasound attenuation and aberration.

Shortcomings of the prior art of record are addressed hereinafter. An insight of the present inventors is the value of taking into account the two-dimensional (2D) nature of the aberration ultrasound experiences upon passing through a temporal bone. For example, part of an approaching ultrasound beam wavefront can be refracted in a lateral direction of 2D space. The direction depends on factors that can include the particular, local surface irregularities, if any, of the temporal bone through which that part passes just before being received by the ultrasound probe.

The instant proposal also addresses the current limitations of microbubble-enhanced stroke therapy, and is aimed at enabling precise control of the therapeutic ultrasound beam profile (especially, the focal location and beam shape) and the ultrasound intensity, i.e., ultrasound exposure dosage.

An inventive device, according to what is proposed herein, includes a two-dimensional array configured for receiving transmissive ultrasound that has passed through an inhomogeneous medium. The device is configured for performing aberration estimation on the received ultrasound such that a result of the estimation is usable in improving ultrasound operation.

In one aspect of the invention, a device such as the above one is configured for modifying, based on the estimation result, a setting of the device so as to at least one of a) improve location of at least one of ultrasound transmission and ultrasound reception; and b) correct beamforming of ultrasound.

In some embodiments, the modifying to improve location is based on selected placement, and/or a selected extent, of an acoustic window.

As to the estimation result, it includes, according to some versions, at least one aberration map for which both elevation and azimuth are independent variables, the modifying being based on one or more of the maps.

From another standpoint, the estimation result includes aberration maps having a spatial independent variable. At least two of signal time delay, signal amplitude, and signal distortion are dependent variables of respective ones of the maps.

In a sub-version, the result includes at least one of a signal amplitude map and a signal distortion map, said device being configured for utilizing at least one of the maps to regulate, as a weighting map, contribution of either individual transducer elements or individual patches to beamforming.

In some aspects, the device comprises a contralateral transducer array and is configured for receive beamforming from both sides from a single ultrasound transmit pulse.

In a sub-aspect, the device is further configured for compounding images acquired on both sides by the beamforming.

For the device, as another sub-aspect, the transmissive ultrasound emanates from the contralateral array. The device is configured such that the beamforming takes into account receive aberration correction respectively based both on the above-mentioned aberration estimation and aberration estimation on contralaterally received transmissive ultrasound.

In a further aspect, the device is configured for emitting, from point sources distributed over a contralateral transducer array, a point source being a patch or transducer element, the transmissive ultrasound, and for, based on the performed aberration estimation, selecting an acoustic window.

In an alternative aspect, the device includes an array placement adjustor configured for translating the two-dimensional array or contralateral array by less than a size of a patch of the array to be translated.

In another aspect, the device includes, for placement contralaterally to the array, a source for the transmissive ultrasound.

In a sub-aspect, the source includes a patch, whose input is initially beamformed separately. The patch serves, for the performing of the aberration estimation, as a point source with respect to the array.

In a different sub-aspect, the source comprises a contralateral array, and the device is configured for focusing, from the contralateral array, a beam on an outer surface of a temporal bone. The focus serves, for the performing of the aberration estimation, as a point source with respect to the transducer array.

In yet another aspect, the ultrasound correction includes tailoring ultrasound to characteristics of a portion of the inhomogeneous medium through which the transmissive ultrasound passes.

In a yet further aspect, a device includes a multi-element transducer array and a display, the device being configured for, based on a result of aberration estimation, predicting a shape of a corresponding aberrated beam, and for displaying, on the display, an image of the predicted shape.

In a sub-aspect, the aberration estimation is performed on transmissive ultrasound that has passed through an inhomogeneous medium and has been received by the transducer array, which is two-dimensional.

An inventive method, according to what is proposed herein, includes receiving, at any given moment, in more than one spatial dimension, transmissive ultrasound that has passed through an inhomogeneous medium; and performing, on the received ultrasound, aberration estimation that correspondingly accounts for aberration laterally in the more than one spatial dimension, a result of the estimation being usable in improving ultrasound operation.

In a specific sub-aspect, the improving includes correcting aberration by modifying phase delays based on a phase delay map having the more than one spatial dimension. Relative time lags between respective pairs of map elements are used in the modifying.

Another method is directed to adjusting ultrasound exposure dosage, and includes providing a contralateral arrangement of transducer arrays. It also includes supplying bubbles to a reference region offset from, but at a depth of, a treatment region. It further includes applying ultrasound in increasing intensity to monitor, by means of at least one of the arrays, increase of amplitude of a subharmonic frequency component of oscillation of the bubbles in relation to increase in the intensity.

In particular other aspects, a device is configured for using a result of aberration estimation on transmissive ultrasound received by a two-dimensional transducer array, to, automatically and without the need for user intervention, modify a setting of the device so as to at least one of a) improve location of at least one of ultrasound transmission and ultrasound reception; and b) correct beamforming of ultrasound.

In yet another aspect, a computer software product enables, through the use of a two-dimensional transducer array to receive transmissive ultrasound that has passed through an inhomogeneous medium, improvement of ultrasound operation. The product comprises a computer readable medium embodying a computer program that includes instructions executable by a processor to perform aberration estimation on the received transmissive ultrasound such that a result of the estimation is usable in the improvement.

As further, additional aspects, devices described above may be implemented as one or more integrated circuits.

Details of the novel, transcranial ultrasound aberration estimation/correction methodology and apparatus are set forth further below, with the aid of the following drawings, in which like structures are annotated by the same or analogous numerals throughout the several views.

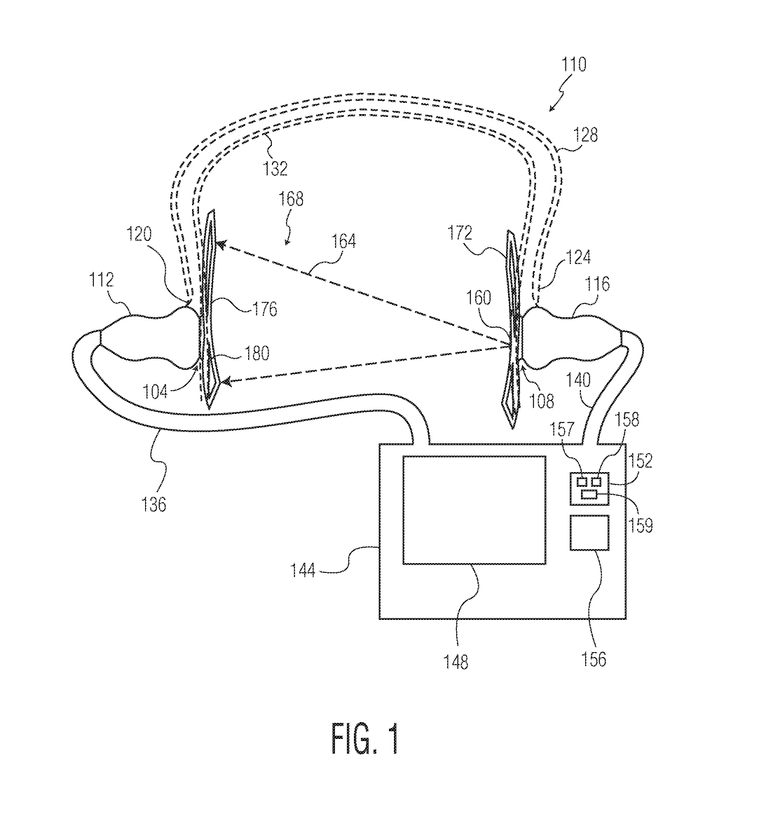

FIG. 1 is a schematic diagram exemplary of contralateral arrangement of 2D ultrasound transducer arrays, a point source of one illuminating a second by means of transmissive ultrasound;

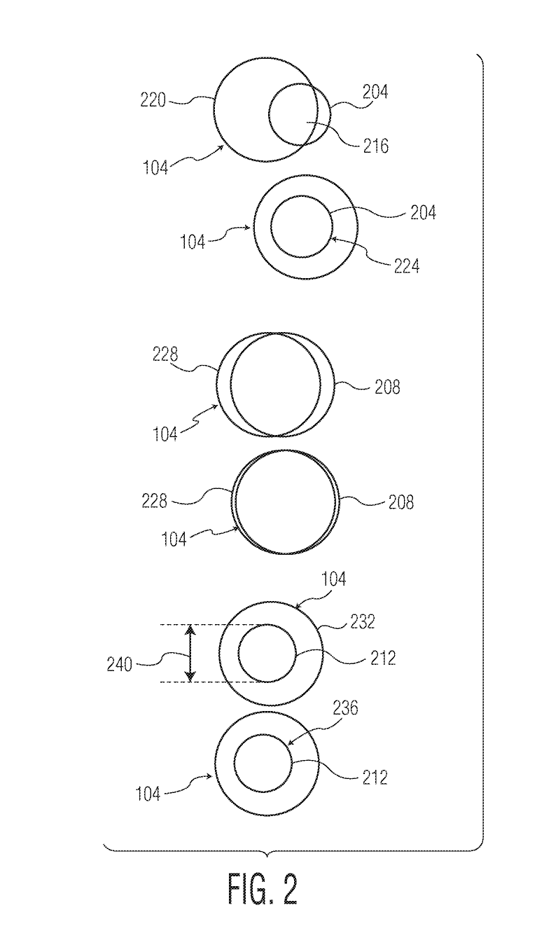

FIG. 2 is a schematic diagram showing examples of selecting acoustic windows based on estimated aberration and of aligning a transducer aperture with the selected window;

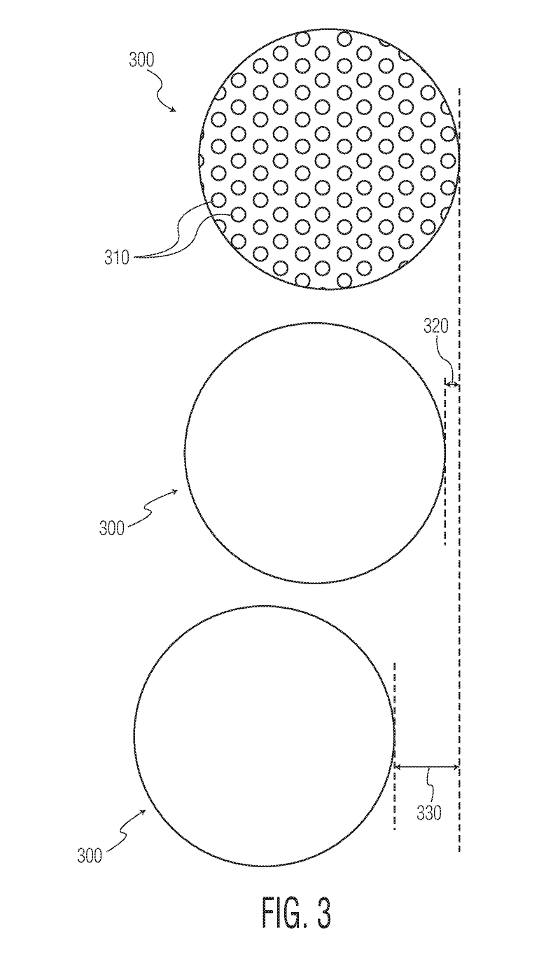

FIG. 3 is a schematic diagram exemplary of a 2D ultrasound transducer array showing its division into patches, and translation of the array to a different position;

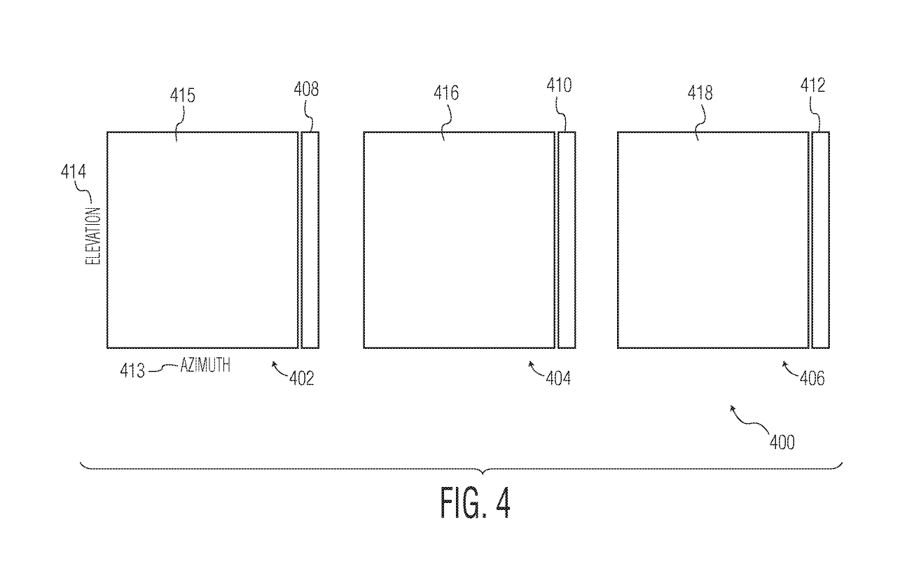

FIG. 4 is graphical depiction of aberration maps derivable by the illuminating in FIG. 1;

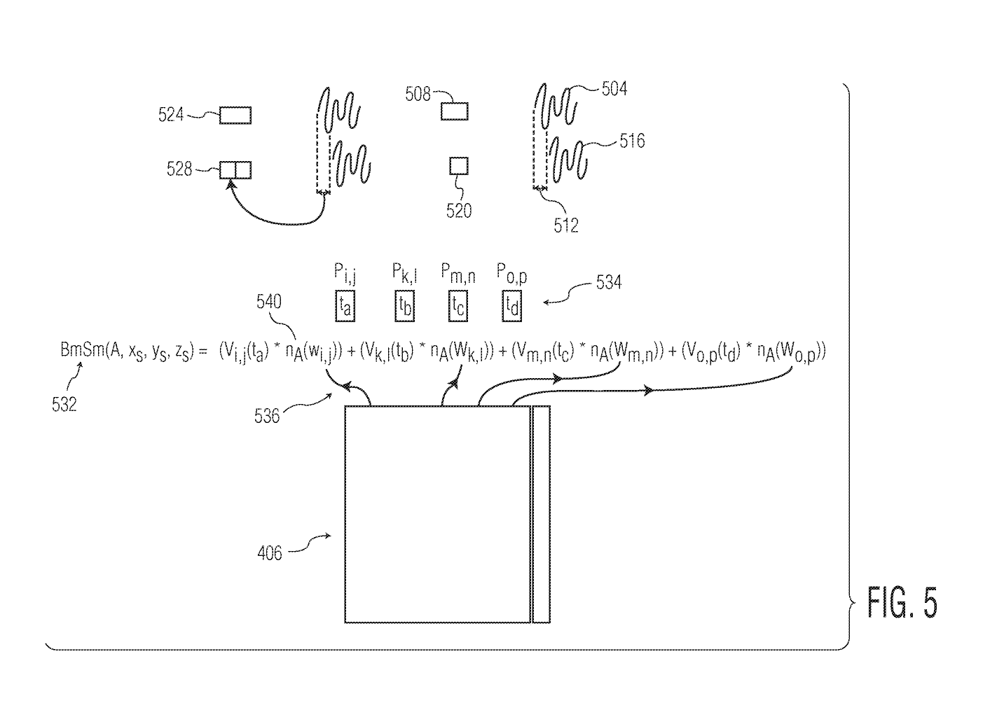

FIG. 5 is a conceptual diagram exemplary of phase delay compensation and of using an aberration map to regulate, as a weighting map, contribution of either individual transducer elements or individual patches to beamforming;

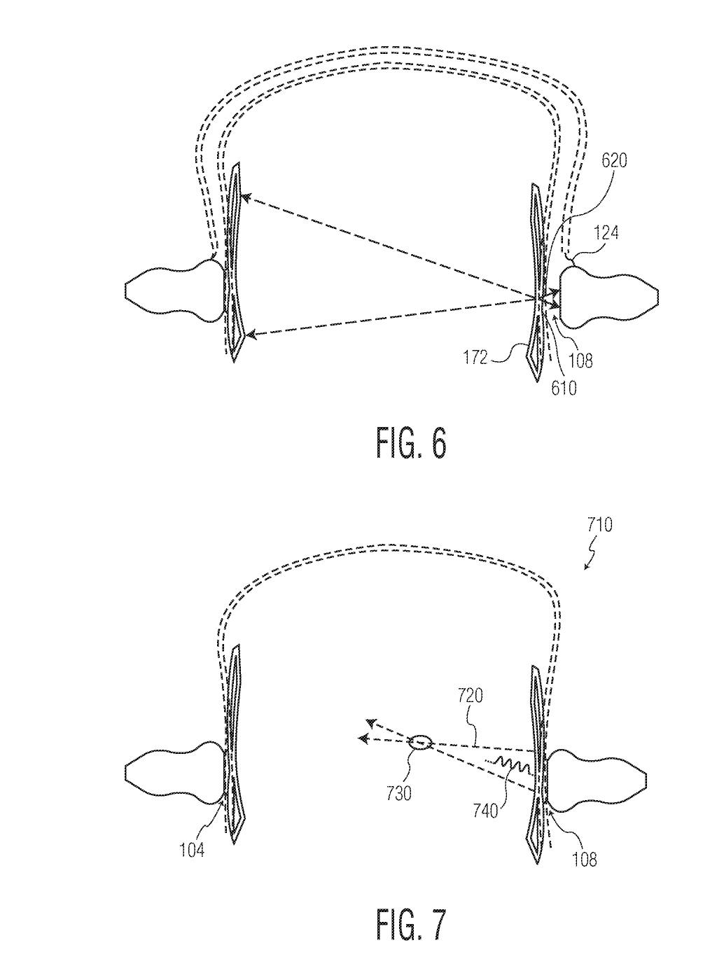

FIG. 6 is an example of a modification of the contralateral arrangement of FIG. 1, in which the transmitting array is translated away so as to focus on the outside surface of the right temporal bone;

FIG. 7 is a schematic diagram of an example of a contralateral arrangement portraying the application of a therapeutic beam to a treatment region;

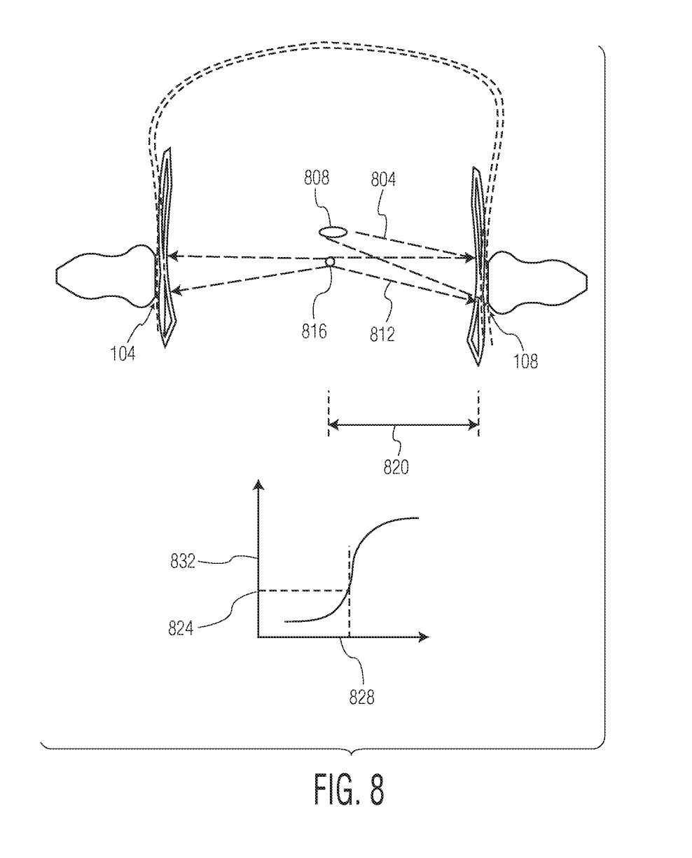

FIG. 8 is a schematic diagram relating to microbubble-based intensity estimation, showing an instance of applying a test beam to a treatment region to measure ultrasound intensity, and another instance of applying a test beam but to a reference region at equal depth;

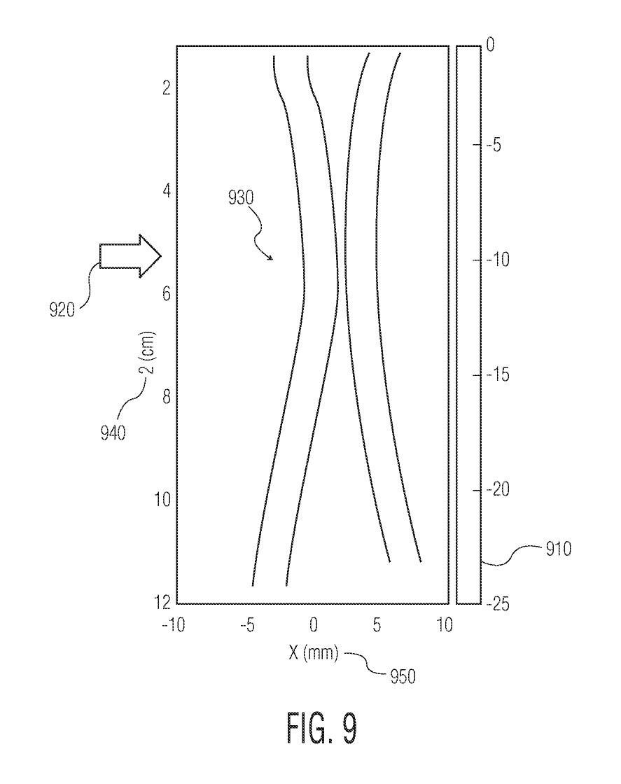

FIG. 9 is a graphical depiction of a possible pattern representative of the predicted shape of a transmit beam taking into account beam aberration; and

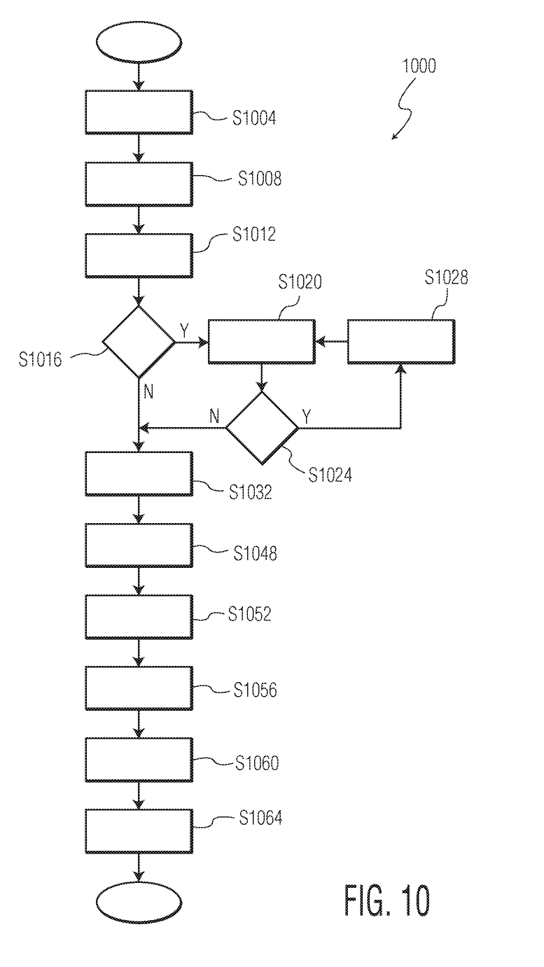

FIG. 10 is flow chart of an exemplary transcranial imaging/therapy aberration prediction/correction process.

FIG. 1 depicts, by way of illustrative and non-limitative example, an ultrasound device 110 having a contralateral arrangement of two-dimensional (2D) transducer arrays 104, 108 housed in respective probes 112, 116. The arrays 104, 108 are respectively connected to array placement adjustors 120, 124. The array placement adjustors 120, 124 are respectively connected to each end of a head frame or head piece 128. The headpiece 128 is supported, by straps, buckles, Velcro® or other adjustable means, fixedly on the skull 132 of the medical subject, such as a human medical patient or an animal, such as a warm-blooded mammal, although the present invention is not limited to any particular living form. The subject could also be a medical sample, in vitro or ex vivo. Each probe 112, 116, is connected by its cable 136, 140 to an ultrasound apparatus 144 which comprises a display 148, a processor 152, and a user control panel 156. The processor 152 can include software 157, and/or one or more integrated circuits 158, and working storage 159, for wave aberration estimation/correction, intensity control, and aberrated-beam profile prediction. Additional potential features of an ultrasound apparatus having a contralateral arrangement of 2D transducer arrays are described in the commonly assigned International Publication Number WO 2008/017997 A2, entitled “Ultrasound System for Cerebral Blood Flow Imaging and Microbubble-Enhanced Blood Clot Lysis,” to Browning et al., the entire disclosure of which is hereby incorporated herein by reference.

Operationally, estimating the aberration that would be encountered in transcranial imaging of or therapy for a particular subject is done in a preliminary procedure. From a point source 160 such as transducer element or patch (i.e., small group of adjacent transducer elements) of the right-hand (or “contralateral”) array 108, a beam 164 of transmissive ultrasound is emitted. The point source 160 can alternatively be a combination of adjacent patches for increasing the acoustic power of the point source. Transmissive ultrasound is ultrasound emitted for reception in the direction of propagation, in contrast to reflective ultrasound which is usually received by the transmitting device. Transmissive ultrasound is also known as ultrasound applied in the through-transmission mode, as opposed to the pulse-echo mode. The beam 164 may be formed by short pulses, e.g., of four cycles each, at for example 3.2 MHz. The beam 164 passes through an inhomogeneous medium 168 which includes a right temporal bone 172 and then a left temporal bone 176 before arriving incident to the left-hand array 104. The term “temporal bone” is sometimes used to denote a single skull bone, but is used herein in the sense of referring to either the left or right temporal bone.

If an aperture larger than a point source is used to emit ultrasound from the right that passes through the right temporal bone 172, surface and shape irregularities of the bone would cause the emerging wavefront to be aberrated. The aperture size is selectable, in relation to the size of the skull 132 and the strength of the aberration induced by the right temporal bone 172, so that the aberrated wavefront becomes regularized by the time it reaches the other side of the skull.

Making the ultrasound source a point source 160, such as a transducer element or patch, virtually eliminates any such aberrating effect in the near field. It is thereby assured that a regular wavefront will approach the other side of the skull 132.

In the far field, the left temporal bone 176 is a portion of the inhomogeneous medium 168 having aberrating characteristics that will come to bear on the ultrasound which arrives incident to the left-hand array 104.

In compensation (after the instant estimating procedure), correcting ultrasound for delivery from the other side, i.e., by means of the left-hand array 104, in the form of a therapeutic or imaging beam aims at tailoring the ultrasound to these characteristics. The tailoring, which may entail phase aberration correction and transmit/receive weighting of transducer elements/patches for beamforming, will be discussed further below in more detail.

Focusing again on the estimating procedure, the receiving, at any given moment, by the left-hand array 104 occurs in two spatial dimensions of the array, so that aberration estimation may correspondingly and advantageously account for aberration laterally in the two spatial dimensions.

For example, the left-hand array 104 receives ultrasound from the point source. 160. Specifically, each receiving element 180, i.e., patch or single transducer element, of the left-hand array 104 samples a series of pressure readings. The readings are recorded as paired values, for that receiving element 180, of amplitude and time of acquisition. This is repeated for the next (adjacent) point source 160, until the last point source is processed.

In certain embodiments, this protocol during aberration estimation is then reversed, with emission from point receivers 180 (now acting as new point sources) of the left-hand array 104, point-by-point, for reception by the right-hand array 108. In other words, the roles are reversed so as to, this time, estimate the aberration characteristics of the right temporal bone 172.

FIG. 2 demonstrates, by example, selecting acoustic windows 204, 208, 212 based on estimated aberration, and the aligning of a transducer aperture with the selected window. The estimated aberration can take the form of aberration maps, which are discussed later in the description.

Based on the estimated aberration, which is available in two spatial dimensions by virtue of an aberration map, an acoustic window 204, 208, 212 is selected.

Firstly, with regard to terminology, the term “temporal window” refers to the ultrasound window afforded by the temporal bone by virtue of its thinness and/or spatial smoothness and consequently minimal attenuating and aberrating affect on ultrasound. The term “acoustic window,” as used herein, also refers to an ultrasound window, and, in some embodiments, to an ultrasound window within the temporal window. More specifically, the acoustic window is the body surface area selected not only for application of the ultrasound transducer 104 but that part of the area for which a transducer aperture will be active. In other words, the acoustic window is the part that is judged, based on the current aberration estimate, to involve the least wave aberration. Because the estimation procedure may be iterative, the terms “best,” “optimal,” and “least aberrating” acoustic window are also used, but all relate to that part of the skull 132 that yields least attenuation, dephasing and waveform distortion compared to the water (or “soft tissue”) path. The acoustic window is generally regarded herein as a continuous area, despite the fact that particular (isolated) points in the area may not receive favorable readings in the aberration estimation.

The first example in FIG. 2 shows the transducer array 104 partially overlapping the acoustic window 204. The estimation procedure has been performed. Based on the current iteration of the procedure, the acoustic window 204 has been selected. The selecting entails selecting at least one of placement and an extent of the acoustic window 204. In this example, a placement 216 can be characterized by a center of the window 204.

By then aligning the array 104 so as to fully encompass the acoustic window 204, an active transducer aperture can fully cover the window under its footprint. Thus, since the acoustic window 204 offers least (or less) wavefront aberration and since an active transducer aperture can now be configured so as to completely cover the window with regard to ultrasound transmission and/or reception, an improvement has been made to the location of ultrasound transmission and/or ultrasound reception. This amounts to improving ultrasound operation, through the use of an aberration estimate.

Moreover, the initial aperture 220, which here included fully the entire array 104, can optionally be customized down to an aperture 224 that matches the acoustic window 204. This constitutes yet another improvement as to location of ultrasound transmission and/or reception, at least because the smaller area entails less ultrasound processing and overhead. This, then, also, amounts to improving ultrasound operation, through the use of an aberration estimate. Here, at least two setting modifications are made to the ultrasound device 110, one being the translation of the array 104 and the other being the reduction of the active transducer aperture. The modifying is based on the estimated aberration, as reflected in the aberration map(s), and is further based here on the placement 216 of the acoustic window 204.

In the second example in FIG. 2, the transducer array 104 happens to be equal in size to the acoustic window 208, which is here again equal in size to the initial active transducer aperture 228. Accordingly, translation of the array 104 to match the acoustic window 208 is performed. However, no resizing or shifting of the aperture 228 is necessary or desirable.

In the third example, there is no partial overlapping of the array 104 with an acoustic window 232; instead, the array already fully encompasses the window. Thus, no translating of the array 104 is needed. The active transducer aperture 232 may advantageously be narrowed to an aperture 236 that matches the acoustic window 212.

What thus has in effect occurred is that based on the current iteration of the estimation procedure, the acoustic window 212 has been selected. The selecting entailed selecting an extent 240 of the acoustic window 212. No device setting modification was required in terms of translating the transducer array 104, because the array already covered the window 212. However, a device setting modification downsized the initial active aperture 232 to a smaller aperture 236.

These are examples of modifying a setting of the device 110 and may be performed interactively.

Although the above examples have been framed in the context of the left-hand array 104, they could equally have been presented with respect to the right-hand array 108. This is due to the contralateral arrangement in which aberrating characteristics are estimated for the left temporal bone 176 and then for the right temporal bone 172, or vice versa.

In some embodiments, the arrays 104, 108 are each divided into patches 310 for improved correction algorithms. FIG. 3 depicts a representative 2D ultrasound transducer array 300 showing its division into patches 310. Each patch 310 is, as mentioned above, a collection of adjacent individual transducer elements. It can be modeled as a small focused transducer in the near field and yet as a point source in the far field. The inputs and outputs of the constituent elements of a patch 310 may be microbeamformed, this being done for each patch in an active aperture (in the aberration correction stage for example). This processing can occur in the probe 112, 116 for instance. A second beamforming stage in the main processor 152 beamforms based on the results for the patches 310 in the aperture. Thus input for the patch 310 is initially beamformed separately, but the results for a plurality of patches 310 are collectively beamformed in a second stage. An example of two-stage beamforming with patches is discussed in more detail in commonly-assigned U.S. Pat. No. 6,623,432 to Powers et al., entitled “Ultrasonic Diagnostic Imaging Transducer with Hexagonal Patches,” the disclosure of which is hereby incorporated by reference herein in its entirety. Although the array 300 is shown here as generally circular, it may be another shape, such as rectangular.

In the above-described aberration estimation procedure, improved resolution is attainable by iteratively slightly translating the receiving, patch-divided array 104 and repeating the procedure. In other words, after estimating aberration, selecting the acoustic window 204, 28, 212, and modifying one or more settings of the ultrasound device 110, the process may be repeated. In this regard, the array placement adjustors 120, 124 are capable of fine lateral adjustment iteratively each time by a distance 320 less than the size of a patch 310 of the adjustor to be translated, for the purpose of fine-tuning resolution.

Moreover, as part of the aberration estimation procedure, the adjustors 120, 124 can handle larger lateral translations 330 made in an effort to find an optimal acoustic window 204, 208, 212.

As discussed further below, the adjustors 120, 124 in some embodiments are further capable of affording or providing movement in the axial direction.

All of the above-mentioned translations or movements may be manual or motorized. If motorized, they may be performed by the ultrasound device 110, based on an estimate of aberration, automatically and without the need for user intervention.

A result of aberration estimation can also be utilized in other interactions based on a display of the shape of a transcranial beam, that shape being predictable by taking into account the aberration estimation result. Those other interactions entail modifications to any of a variety of other settings of the device 110 and are likewise discussed in more detail further below.

FIG. 4 graphically portrays three examples of aberration maps 400 upon which ones of such interactions may be based. The aberration maps 400 are derivable by means of the source point-to-receiving array aberration estimation procedure discussed in connection with FIG. 1. The aberration maps 400 portrayed are a (signal) phase delay map 402, a (signal) amplitude loss map 404, and a (signal) waveform distortion map 406. To the right of each map 402, 404, 406, is the corresponding scale 408, 410, 412. Both the maps 402, 404, 406 and their scales 408, 410, 412 are, in a continuous spectrum, color-coded, although seen here in black and white. Thus, for example, the top portion of the phase delay map scale 408 is colored differently from the bottom portion, this being indistinguishable in the black and white graph shown in FIG. 4. This being said, the design and functions of the maps are believed to be demonstrable from the black and white graphs shown.

All three maps 402, 404, 406 have spatial independent variables, i.e., independent variables in a spatial dimension. For each map 402, 404, 406, their horizontal dimension is azimuth 413 and their vertical dimension is elevation 414. Azimuth 413 and elevation 414 are the (spatial) independent variables. Phase delay, amplitude loss and waveform distortion are dependent variables of the respective maps 402, 404, 406.

Physically, the axial direction is normal to the face of the transducer array 104, 108, i.e., into the skin. The azimuthal direction is lateral, from side to side, and the elevation direction is up and down.

The three maps 402, 404, 406 are, accordingly, mathematical arrays, each element 415, 416, 418 of the respective map corresponding to an associated receiving element or patch 180 from which amplitude versus time samples are acquired and stored.

The samples are of ultrasound pressure which is modeled for a given map element 415, 416, 418 as a sinusoidal input waveform or trace.

The elements 415 of the phase delay map 402 are temporal, i.e., time, delays which may be expressed in microseconds. The temporal delays are element-wise relative to one another. Sound travels faster through bone than through soft tissue. For a given point source 160, a portion of an ultrasound wave that passes through a relative thin part of the temporal bone 172, 176 and is incident upon its respective receiving element 180 will, other factors being equal, tend to arrive later than another portion that passes through a thicker part of the temporal bone. The relative lead/lag constitutes an aberration of the ultrasound wavefront which, if not accounted for or corrected, would potentially introduce error into the therapeutic or diagnostic application of ultrasound.

Even in the case of a regular, unaberrated wavefront arriving at the receiving array 104, 108 from a contralateral point source 160 in the far field, the arriving wavefront would be spherical and centered upon the point source; accordingly, the receiving elements 180 generally differ as to their respective distances from the current point source. To back out this geometrical effect not representative of aberration, the waveforms associated with the receiving elements 180 are, initially, aligned. The alignment is based on a homogeneous speed of sound. Thus for example, if, due to geometry, one waveform travels a longer distance than another, the distance is divided by a speed of sound that is common for all such calculations of one waveform to another, in determining an aligning time shift for a waveform.

Once the waveforms of each receiving element 180 are aligned, processing can proceed either in the time domain or the frequency domain.

In the time domain, one embodiment may be the following: cross-correlation searches are performed between pairs of waveforms. First, a “total beam sum” signal is calculated by summing coherently all of the waveforms, i.e., one added per each receiving element 180. The total beam sum signal serves as a reference waveform. A cross-correlation search is performed between the reference waveform and the waveform of a receiving element 180. This is done for each receiving element 180. So, if there are N receiving elements 180, N cross-correlation searches are performed. Each cross-correlation search yields a respective time lag, which provides the temporal delay value in the associated element 415 of the phase delay map 402. To somewhat simplify the map calculation, a receiving element 180 centrally located in the array 104, 108 can be chosen, and its waveform, instead of the total beam sum signal, can serve as the reference waveform. This is based on the idea that the central location exists over the thinnest part of the temporal bone 172, 176 and consequently experiences the least attenuation and waveform distortion. A further alternative, more robust at the expense of extra computation, is to perform, after waveform alignment, cross-correlation searches between each combinatorial pair of waveforms, i.e., N*(N−1) searches if there are N elements 180. The result is N*(N−1) differential time values. This set of values can be inverted to yield N “absolute” time values, which are not really absolute but determined up to a constant value, which for practical purposes does not matter.

To proceed, instead, in the frequency domain, the geometrically aligned waveforms are Fourier-transformed in the temporal dimension. In the way of background, we start with the fact that, from a point source 160 in the above-described aberration estimation procedure of FIG. 1, the beam 164 emitted is formed from one or more propagating short pulses. This pulse contains a certain range of frequencies around the central frequency, which is the frequency of the modulated sine wave. So, several frequencies are acquired by sending a single pulse. Each frequency component of the pulse has an amplitude and a phase. The shorter the pulse, the wider the frequency range that is sent out. In accordance with Fourier decomposition, the pulse is the sum of a number of continuous sinusoids of different frequencies. Each sinusoid has an amplitude and a phase.

The aligned waveform inputted to the Fourier transform is an “amplitude versus time” sequence. The output is a sequence of frequencies each of which is associated with a particular amplitude and a particular phase. These frequencies are of the above-discussed frequency components, the transformation yielding the particular amplitude and phase. Each of the aligned waveforms is transformed to yield the same sequence of frequencies. With each frequency, an amplitude and a phase both particular to the waveform are determined.

Next, the phase delay per element 415 is extracted, these forming a phase delay map. More specifically, any given one of the aligned waveforms is the input of a corresponding receiving element 180. Each receiving element 180 is associated with a respective element 415 of the phase delay map 402 which ultimately is to be formed.

Accordingly, extracting, for a given frequency, the phase yielded per waveform by the transformations creates a phase map for that frequency. These phase maps are phase-unwrapped. Phase unwrapping, in this context, is a known mathematical procedure for ensuring that there are no artificial phase discontinuities between adjacent elements. In each of the resulting phase maps, one per frequency, the phase is divided by angular frequency and is thereby converted into a temporal delay.

The phase-unwrapped, converted maps are then averaged, weighting each by the amplitude of the transducer's spectrum at the corresponding frequency. The frequency-based amplitudes being utilized as weights may be acquired in the waveform acquisitions described above; or instead, they may be values characteristic of the source transducer, each being the amplitude with which the corresponding frequency is received by the electronics.

The weighted average, element-by-element, results in a single map, i.e., the phase delay map 402.

The phase delay map 402 may be produced separately for each point source 160, by, for example, turning on one patch 310 after another in sequence. If, therefore, N points sources 160 are utilized, N phase delay maps are available for analyzing aberration based on the adjacent temporal bone 172, 176. Repeating the procedure contralaterally, i.e., by reversing the source and destination of ultrasound, yields N more phase maps if there are N contralateral point sources 160, this second set of N maps for analyzing aberration based on the other temporal bone 172, 176.

In order to enhance robustness of the phase delay map estimate, these N delay maps 402 may be averaged. Each delay map in the average is weighted by the corresponding measured waveform attenuation suffered through the skull by the signals emitted by each corresponding source point 160. The weights may correspond to the elements 416 of the contralaterally produced amplitude loss map 404, i.e., produced from transmissive ultrasound in the opposite direction.

In an alternative version, one of the arrays 104, 108 may be replaced with a small-aperture, single-element transducer 160 as a point source which is physically scanned from point source location to point source location. The arrangement may then be physically reversed for analyzing the contralateral temporal bone 172, 176.

Before discussing more on how the phase delay maps 402 may be used, the two other types of aberration maps 404, 406 shown in FIG. 4 will be explained.

For the amplitude loss map 404, for a given receiving element 180, extraction is made of the temporal maximum of the received waveform. The waveform is in the form of amplitude as a function of time, so that the temporal maximum is an amplitude. This is done for all receiving elements 180 (or, equivalently, for all map elements 416). The resulting 2D map of amplitudes is normalized by its maximum. In other words, each amplitude is divided by the maximum over all the amplitudes of the map. The resulting values are each converted to decibels by taking the base 10 logarithm and multiplying by 20. A −6 dB reduction in amplitude, for example, is accordingly a reduction by about 50%.

In forming the waveform distortion map 406, for each element 418 the waveform is compared to a reference waveform. The reference waveform is acquired, typically beforehand in a non-clinical setting, in a similar contralateral arrangement around an inhomogeneous medium in the absence of skull bone. The comparison just mentioned involves delaying and scaling the reference waveform so that it overlaps as well as possible the first few cycles of the waveform whose distortion is being measured. A metric for distortion of the waveform can be expressed as:

$$m = \frac{{\int }_{ }^{ } s ( t ) {s}_{ref} ( t ) d t}{{\int }_{ }^{ } {s ( t )}^{2} d t} \qquad equation ( 1 )$$

where s_{ref} (t) is the delayed and scaled reference waveform, and s(t) is the waveform whose distortion is being measured.

The metric equals one if there is no wave distortion s(t)=s_{ref}(t) and tends to zero if there is a strong waveform elongation, for instance due to in-skull or transducer-skull reverberations.

The utility of the waveform distortion maps 406 resides in the fact that waveforms with well-controlled bandwidths (e.g., with Gaussian envelopes) should be transmitted so that the influence of brain tissue attenuation on waveform distortion can be minimized.

As mentioned above in connection with the phase delay map 402, the aberration maps 402, 404, 406 can be generated point source by point source, and contralaterally in reverse so as to account for aberration due to the contralateral temporal bone 172, 176.

Point sources 160 on the same side afford different angles of approach to a given contralateral receiving element 180 and correspondingly different angles of incidence with a potentially irregular surface of the temporal bone 172, 176 adjacent that contralateral receiving element. Accordingly, even a small differential as to angle of approach can significantly vary one map from another on the same side. Also, thickness variations in the near field temporal bone 172, 176 may cause one of the maps to be based on a significantly higher signal-to-noise ratio (SNR) than another on the same side, hence the interest of combining (e.g. in a weighted average) several maps obtained with several contralateral elements to enhance the quality of the estimate of the final aberration maps.

The aberration maps 402, 404, 406 are usable in improving ultrasound operation, such as that achieved by improving the location of ultrasound transmission and/or reception and/or by correcting the beamforming of ultrasound.

The phase delay map 402 can for instance be used to correct temporal misalignment of received signals due to the crossing of the inhomogeneous skull 132, by modifying receive beamforming delays. This is an example of receive aberration correction. The phase delay map 402 is consulted for those elements 415 within the receive aperture, and receive beamforming delays are modified to compensate for relative delays associated with those elements, thereby correcting the receive ultrasound beam line. Likewise, as mentioned further above, knowing the relative time delays allows correction of a transmit beam, through modifying transmit beamforming delays.

FIG. 5 depicts conceptually one example of phase delay compensation and of using an aberration map to regulate, as a weighting map, contribution of either individual transducer elements or individual patches to beamforming. These are examples of tailoring ultrasound to characteristics of a portion 176 of the inhomogeneous medium 168 through which the transmissive ultrasound passes. The characteristics are reflected in the aberration maps 402, 404, 406. They are then reflected in the selection of an acoustic window 204, 208, 212 and/or in the correction of beamforming. That correction can take the form of phase delay adjustment and/or diminishing/increasing the individual contributions of transducer elements/patches to beamforming.

A first waveform 504 which represents reception of an ultrasound wavefront by one transducer array element 508 leads, by a time lag 512, a second waveform 516 similarly representing reception by a second element 520. Here, it is assumed that the time lag 512 is due to aberration and not to geometry. In other words, it is assumed in this example that the two waveforms 504, 516 have been geometrically aligned. Accordingly, the time lag 512 is derivable from the difference between the corresponding elements 415 of the phase delay map 402. For a given aperture and field point, and before taking into account the time lag 512, e.g., before the current ultrasound emanated, the first waveform 504 would have been assigned a particular reception delay 524. The second waveform 516 would have been assigned its particular reception delay 528. However, taking into account the time lag 512 as an aberrating dephasing of the two waveforms 504, 516, the second delay 528 is increased by the time lag, to thereby remove the aberration-based phase error. Analogously, the same time lag 512 is applied in transmit beamforming. Accordingly, based on the phase delay map 402 having two spatial dimensions, relative time lags 512 between respective pairs of map elements 415 are used to modify delays, so that phase delay based aberration correction is thereby performed.

These are instances of modifying a setting of the device 110, a beamforming delay in particular, to correct beamforming of ultrasound. The modifying is based on an estimate of aberration and, more directly, upon an aberration map 402 which is a result of the aberration estimation.

The other two aberration maps 404, 406 can assist in the beamforming correction process. This assistance is in the form of either diminishing or enhancing the contribution of, as the case may be for the associated array 104, 108, either individual transducer elements or individual patches.

Aside from the fact that beamforming is done dynamically in receive but is static on the transmit, the two forms of beamforming are performed in a similar manner.

Considering first the case of receive beamforming and referring again to FIG. 5, patches P_{i,j}, P_{k,l}, P_{m,n}, P_{o,p} make up a receive aperture A. A field point (xₛ, yₛ, zₛ) is a point in the ultrasound subject, e.g., patient, from which a particular ultrasound echo which is to be measured returns. The measuring occurs by means of the patches P_{i,j}, P_{k,l}, P_{m,n}, P_{o,p} 534 to which the echo returns. Respective samples taken at geometrically-derived times tₐ t_{b} t_{c} t_{d} each give a different “take” on the acoustic reflectivity at the field point. Accordingly, the samples, in the form of voltage amplitudes v_{i,j}(tₐ), v_{k,l}(t_{b}), v_{m,n}(t_{c}), v_{o,p}(t_{d}) representative of acoustic pressure are added to obtain a more robust and spatially more complete view of the reflectivity. The sum is known as a “beamsum” 532. It is a function of the aperture A and of the field point (xₛ, yₛ, zₛ). To correct for waveform distortion, a weighted sum is used, instead of a simple sum. For weights w_{i,j}, w_{k,l}, w_{m,n}, w_{o,p}, the corresponding entries 417 of the waveform distortion map 406 are usable. This is represented by the flow arrows 536 from the distortion map 406, as seen in FIG. 5. To maintain imaging brightness, the weights w_{i,j}, w_{k,l}, w_{m,n}, w_{o,p} may be normalized to unity, so that, for example, their average is one. This yields the weights n_{A}(w_{i,j}), n_{A}(w_{k,l}), n_{A}(w_{m,n}), n_{A}(w_{o,p}) for the aperture A. The resulting beamsum 532 is:

BmSm(A,xₛ,yₛ,zₛ)=(v_{i,j}(tₐ)*n_{A}(w_{i,j}))+(v_{k,l}(t_{b})*n_{A}(w_{k,l}))+(v_{m,n}(t_{c})*n_{A}(w_{m,n}))+(v_{o,p}(t_{d})*n_{A}(w_{o,p})) equation (2)

Utilizing this beamsum, output of the receiving patches P_{i,j}, P_{k,l}, P_{m,n}, P_{o,p} that have been found, by virtue of the distortion map 406, to suffer greater distortion contributes less to focusing. Specifically and by way of example, n_{A}(w_{i,j}) represents the contribution 540 of the transducer element in the i^{th} row and j^{th} column to receive beamforming with respect to the field point (xₛ, yₛ, zₛ) by means of the aperture A for the sample acquisition timing tₐ t_{b} t_{c} t_{d}.

Weighing the contributions 540 by viability of the patch input improves ultrasound operation, and is accomplished by modifying a setting of the ultrasound device 110. The modified setting, here, is a voltage amplitude weight n_{B}(w_{i,j}), nᵤ(w_{k,l}), n_{B}(w_{m,n}), n_{B}(w_{o,p}) for the transmit aperture B.

The weights n_{B}(w_{i,j}), n_{B}(w_{k,l}), n_{B}(w_{m,n}), n_{B}(w_{o,p}) are usable in transmit beamforming, in weighting the voltage levels v_{i,j}(tₐ), v_{k,l}(t_{b}), v_{m,n}(t_{c}), v_{o,p}(t_{d}) to be applied in driving the patches P_{i,j}, P_{k,l}, P_{m,n}, P_{o,p} of the transmit aperture B, which typically is the same as the receive aperture A.

An alternative is to use the amplitude loss map 404 as the weighting map. The map 404 could also be used to apply a “matched filter” on the amplitudes. Specifically, it is assumed that signals from or to transducer elements/patches corresponding to map elements 416 of relatively low value cross rough portions of the skull 132 and negatively affect the focusing quality. Those transducer elements/patches are accordingly, on transmit, driven with even a lower power, and/or, on receive, weighted downwardly in the beamsum, to thereby diminish their relative contribution 540 to transmit/receive beamforming.

As set forth above, the amplitude loss map 404 and the distortion map 406 are both separately utilizable for selectively compensating and/or diminishing per-element power-driving levels on receive or on transmit. Alternatively, a combination of the two maps 404, 406 can be used.

As another possibility, low values of the amplitude loss map 404 can be accordingly amplitude-compensated, by increasing power levels on transmit and weights on receive, so that all elements contribute equally to the focusing.

The ultrasound device 110 is, as set forth above, configured for utilizing at least one of the amplitude and distortion maps 404, 406 to regulate, as a weighting map, contribution 536 of either individual transducer elements or individual patches to beamforming.

Selecting the (best) acoustic window 204, 208, 212 can be based on any of the aberration maps 402, 404, 406. Areas of low amplitude loss, low waveform distortion, and long time-of-flight (corresponding to a shortest path through the high speed-of-sound bone) indicate presence of the thinnest bone and the best acoustic window, for imaging or transtemporal energy deposition for example.

Thus, in the case of the phase delay map 402, the entries 418 of largest amplitude, i.e., largest temporal delay, are indicative of the best acoustic window. This is judged map by map, because the delay values are biased by the thickness of the temporal bone 172, 176 at the contralateral source point 160.

One, two or all three maps 402, 404, 406 can be used to optimize placement of the probes 112, 116 on the temporal bones 172, 176 in front of the best acoustic windows in an automatic way, even without need for user intervention, or by providing visual feedback to the ultrasound user by which the user can manually or by motorized means reposition the probes.

It may be preferable to use aberration maps 402, 404, 406 derived based on respective frequencies. The phase map 402, as noted above, is created as a weighted average of phase maps for respective frequencies. The amplitude and distortion maps 404, 406, too, can be produced separately by frequency, i.e., the center frequency of the received ultrasound. These frequency specific maps 402, 404, 406 are usable for optimal performance at the frequency used during operation. In particular, slight frequency-based variation in the selected acoustic window will generally imply concomitant adjustment to array translation and/or beamforming correction.

A further alternative exists to sequentially scanning the point source 160. The arrays 104, 108 are both retained, but the point source 160 is not scanned consecutively. Instead, a number of point sources 160, generally not consecutive or not all consecutive are fired together to enhance SNR. Several schemes can be used, including the use of spatial (e.g. Hadamard) and temporal (e.g. chirps) encoding, and use of focused beams from the array on the right (these can be converging or diverging beams, and the focus could be inside or outside the brain). Here, before doing any of the signal processing on the received waveforms, i.e., in the time or frequency domain, the received signals are inverted so as to reconstruct the signals that would have been obtained with contralateral sources 160 that would be as close as possible to the surface of the temporal bone 172, and that would be fired one by one. This is known as spatial decoding. An example of spatial encoding is Hadamard coding. If, for example, there are four point sources 160 on the other side of the skull 132, it may be decided to fire them sequentially according to the sequence: 1 0 0 0 0 1 0 0 0 0 1 0 0 0 0 1 or the following Hadamard sequence can be used: 1 1 1 1 1 1 −1 −1 1 −1 −1 1 1 −1 1 −1 in which 1 represents “on”, −1 represents “on” with inverted phase, and 0 represents “off”. The receive signals are manipulated to recreate the ones that would have been obtained with the first, i.e., one point source at a time, sequence. The SNR is enhanced here by using several transducers to transmit at one given time. The point sources 160 for Hadamard coding are distributed over the transmitting transducer array 104, 108, as with one-point-source-at-a-time firing. There are other known, alternative spatial coding schemes that can be utilized.

FIG. 6 demonstrates modification of the contralateral arrangement of FIG. 1, in which the right-hand array 108 is translated away so as to focus on the outer surface 610 of the right temporal bone 172. The right-hand array 108 is placed at a short distance from the right temporal bone 172 so that its beam focus 620 is employed as a virtual point source on the outer surface 610. In this way, the beam transmission loss through the right temporal bone 172 can be calculated based on the reflected signals received by the right-hand array 108. This makes it possible to measure the transcranial transmission coefficient and further predict ultrasound intensity inside the brain. Predicting intensity is done in preparation for applying a therapeutic beam, such as a high-intensity focused ultrasound (HIFU) beam. The right-hand array placement adjustor 124 is shown in an axially extended position. This position may be reached manually or through motorized displacement. It may, for example, be achieved interactively through display on the apparatus display 148 or by means the intensity of the received reflected signal. If motorized, the displacement may be performed by the ultrasound device 110, based on the intensity for example, automatically and without the need for user intervention. A contact medium such a gel pillow is maintained to provide a continuous ultrasound propagation path in the extended position.

FIG. 7 is a schematic diagram of an example of a contralateral arrangement 710 portraying the application of a therapeutic beam 720 to a treatment region 730. The transcranial aberration of the therapeutic beam 720 can be corrected using the aberration maps 402, 404, 406, according to the discussion above.

The therapeutic beam placement is visualized on the display 148 by applying dynamic receive focusing beamforming from both arrays 104, 108. In particular, scattered/reflected signals from the incident therapeutic beam 720 are received by both arrays 104, 108 of the contralateral arrangement of device 110, and beamformed with 3D dynamic focusing in receive. Thus, the ultrasound device 110 may be configured for receive beamforming from both sides from even a single transmit ultrasound pulse 740, and from a series of transmit pulses. Receive beamforming by the non-transmitting array 104 can be likened to perceiving in a given instant, in fog, the headlights of a vehicle traveling generally toward you but headed toward one side or the other.

Receive beamforming can include taking into account receive aberration correction based on the previously acquired aberration maps 402, 404, 406 of the temporal bones 172, 176 underneath the probes' footprints. Phase aberration correction, for example, can be part of the receive beamforming. The correction could have been made in modifying a setting, such as a patch weight, of the ultrasound device 110. Alternatively, it can, incident to beamforming, be dynamically made based on a previous modification, in each case the modification having been made based upon a result of aberration estimation.

While the therapeutic beam 720 is maintained with the same transmit beamforming parameters, two contralateral “single transmit” images are continuously obtainable from both arrays 104, 108 locked on the temporal bone windows.

Enhanced visualization, in real time, of the location and extent of the beam 720 is attained by compounding the two images, means for compounding two images being well-known in the art.

The therapeutic beam visualization will guide the adjustment of the focal position and size of the therapeutic beam 720. The visualization can also be enhanced by receiving sub- or super-harmonics from contrast microbubbles in case of their presence.

FIG. 8 relates to microbubble-based intensity estimation, showing an instance of applying a test beam 804 to a treatment region 808 to measure ultrasound intensity, and another instance of applying a test beam 812 but to a reference region 816 at equal depth 820.

Microbubble-based ultrasound contrast agents are often used in ultrasound-mediated or ultrasound-enhanced stroke therapy because vibrating microbubbles next to a clot (causing arterial occlusion and inducing ischemic stroke) can significantly increase the local ultrasound exposure to the clot. Ultrasound intensity in the treatment (or occlusion) region can be estimated by measuring the thresholds for onset of subharmonic emission from contrast microbubbles within the treatment region 808, or from within a reference region 816 close to the treatment region. The use of a reference region 816 rather than the treatment region 808 for measurement of cavitation onset is motivated by the need for adequate flow and/or perfusion of contrast microbubbles in order to receive robust signal from insonified microbubbles. As an example, the reference region 816 is shown next to the treatment region 808 but at the same depth 820 (so that ultrasound attenuation from any of the probes 112, 116 to the reference region is similar to the attenuation from that probe to the treatment region).

The subharmonic signal onset 824 in the treatment or reference regions 816, which varies with the contrast agent used, can be determined by gradually increasing the intensity (or acoustic pressure) 828 of the test beam 804, 812 until robust subharmonic signals, whose amplitudes 832 are shown in FIG. 8, are (suddenly) received by the left-hand array 104 or the right-hand array 108. Accordingly, increase of the amplitude 832 of a subharmonic frequency component of bubble oscillation in relation to increase in intensity 828 is monitored via the arrays 104, 108 to detect the sudden onset of stable cavitation.

Measurement of subharmonic signal amplitude versus acoustic pressure, taken in a non-clinical, experimental setting, is discussed in U.S. Pat. No. 6,302,845 to Shi et al., entitled “Method and System for Pressure Estimation Using Subharmonic Signals from Micro-Bubble Based Ultrasound Contrast Agents.” More measurement details are given in the reference “Shi W T, Forsberg F, Raichlen J S, Needleman L, Goldberg B B. Pressure dependence on subharmonic signals from contrast microbubbles. Ultrasound Biol Med 1999; 25: 275-283”. The entire disclosure of both documents is hereby incorporated herein by reference.

As set forth above, microbubble-enhanced stroke therapy is improved by more precise placement and by intensity prediction for a therapeutic beam.

A further beneficial feature is the ability to predict the shape of an aberrated therapeutic beam based on estimated aberration and the transmit beamforming parameters, and the possibility to interactively adjust the transmitted beam to reduce aberration.

FIG. 9 depicts a possible pattern 910 representative of the predicted shape 920 of a transmit beam 930 taking into account beam aberration.

The pattern 910 is an example of what is displayed to the user as the prediction 920 of the shape of the ultrasound beam 930 to be applied, e.g., a therapeutic beam. In this figure, the vertical axis (z) in centimeters is in the axial direction 940, and the horizontal axis x in millimeters is in the azimuth direction 850. In practice, 2D beam profiles (axial*azimuth, or axial*elevation) or 3D beam profile may be displayed. Here, the beam focus is at about approximately 5 centimeters. The scale strip on the right represents relative temporally average intensity levels. Again, the legend was originally produced in color, but is shown here in black and white. In particular the intensity values are normalized based on their maximum value (over the entire space being depicted) and displayed in decibels. The function need not be temporally average intensity, but could, instead, be, for example, the temporal maximum of the pressure amplitude, or the mechanical index (MI).

The capability to predict beam shape based on transmit beamforming parameters and estimated aberration is particularly advantageous for imaging media for which aberration is known to be a significant problem, but also is suited generally as a tool in the therapeutic use of ultrasound.

In some embodiments, a multi-element transducer array 104 receives ultrasound, software or hardware estimates aberration, and software predicts the aberrated ultrasound beam shape, an image of which is then displayed.

Specific techniques for predicting beam shape based on beamforming parameters and on the aberration estimate are set forth in the discussion below with the simplified example of a 1D array for 2D imaging and therapeutic beam steering. These techniques can easily be generalized to a 3D setting.

Let us assume that the aberrator, e.g., the temporal bone, is infinitely thin and infinitely close to the measuring array. Then the aberration can be described in terms of a phase (shift) and amplitude (attenuation) per element per frequency. Means for measuring this aberration have been described in connection with the phase delay map 402. So, the aberration map Ab(x,ω) in 1D along spatial dimension x and at angular temporal frequency ω can be written in the following form:

A_{Ab}(x,ω)=A(x,ω)e^{iφ(x,ω)} (a)

A(x, ω) being the amplitude (attenuation) term and φ(x , ω) the phase (aberration) term. Now, say that our imaging or therapy device is affected with such aberration. We still want to focus at a certain depth and azimuth in the medium and we are doing it with a certain transmit apodization A_{Apod} (x). Which, means, we program the transmit beamformer to send out the following wavefront

A_{Foc}(x,ω)=A_{Apod}(x)e^{iθ(x,ω)} (b)

θ(x,ω) being the geometrical (cylindrical in 1D arrays, spherical in 2D arrays) focusing phasing necessary to focus at the desired location in the medium (e.g., on a blood-vessel-occluding clot). For focusing at depth z₀, azimuth x₀, the transmit phasing is (c is the speed of sound)

$$θ ( x , ω ) = - \frac{ω}{c} \sqrt{{( x - {x}_{0} )}^{2} + {z}_{0}^{2}} \qquad ( c )$$

Because of the aberration, what is really penetrating the brain is the following wavefront:

Aₛₑₙₜ(x,ω)=A_{Foc}(x,ω)A_{Ab}(x,ω)=A_{Apod}(x)A(x,ω)e^{iφ(x,ω)+θ(x,ω)} (d)

1. Rayleigh-Sommerfeld Beam Prediction

The Rayleigh-Sommerfeld equation teaches us directly what the field A_{fieid}(x_{f}, z_{f}, ω) should be at any point (x_{f}, z_{f}) in the medium based on Aₛₑₙₜ(x,ω):

$${A}_{field} ( {x}_{f} , {z}_{f} , ω ) = \frac{ω}{2 π} {\int }_{ }^{ } d x \frac{{z}_{f}}{{r ( x , {x}_{f} , {z}_{f} )}^{2}} {A}_{sent} ( x , ω ) {e}^{i \frac{ω}{c} r ( x , {x}_{f} , {z}_{f} )} \qquad ( 1 )$$

With r(x, x_{f}, z_{f})=\sqrt{(x−x_{f})²+z_{f}²} being the distance between any array element (at azimuthal position x) and the field point (x_{f}, z_{f}) where we want to determine the field. The integration domain is the array aperture.

The following formula is often taken as a simpler version of formula (I) for simple sources, while keeping a good approximation in practical cases:

$${A}_{field} ( {x}_{f} , {z}_{f} , ω ) = \frac{ω}{2 π} {\int }_{ }^{ } d {xA}_{sent} ( x , ω ) {e}^{i \frac{ω}{c} r ( x , {x}_{f} , {z}_{f} )} \qquad ( 2 )$$

In summary, predicting the field at any point given the measured aberration A_{Ab}(x,ω) and the known applied transmit wavefront A_{Foc}(x,ω) involves:
Multiplying the aberration to the transmit wavefront to obtain Aₛₑₙₜ(x,ω)=A_{Foc} (x,ω)A_{Ab}(x,ω), the wavefront effectively sent into the medium (eq. (d))
Inputting Aₛₑₙₜ(x,ω) into the Rayleigh-Sommerfeld integral (eq. (1) or (2)).
In order to know the temporal field received in the medium, an inverse temporal Fourier transform of the computed field A_{field} (x_{f},z_{f},ω) is performed.

2. Fourier or “Angular Spectrum” Repropagation

The sent field Aₛₑₙₜ(x,ω) can be decomposed into its angular spectrum components by taking its lateral (spatial) Fourier transform.

Aₛₑₙₜ(x,z=0,ω)=∫dkₓU(kₓ,z=0,ω)e^{ik}^{ₓ}^{x} (1)

Similarly, the field sensed at depth z (that we want to predict based on the sent field Aₛₑₙₜ at z=0) can be decomposed as:

A(x,z,ω)=∫dkₓU(kₓ,z,ω)e^{ik}^{ₓ}^{x} (2)

The following relationship exists between the angular spectra at depth z and depth 0, respectively:

$$U ( {k}_{x} , z , ω ) = U ( {k}_{x} , z = 0 , ω ) {e}^{i \sqrt{{( \frac{ω}{c} )}^{2} - {k}_{x}^{2}} z} \qquad ( 3 )$$

In summary, getting the field at depth z from the measured aberration and the known, applied transmit wavefront entails:
Multiplying the aberration to the transmit wavefront to obtain Aₛₑₙₜ(x,ω)=A_{Foc}(x,ω)A_{Ab}(x,ω) the wavefront effectively sent into the medium (eq. (d));
Fourier-transforming Aₛₑₙₜ(x,ω) over the lateral dimension in order to get the angular spectrum U(kₓ,z=0) (eq. (1));
Propagating the angular spectrum in order to get U(kₓ,z,ω) (eq. (3)); Inverse-Fourier-transforming U(kₓ,z,ω) to get the field A(x,z,ω) at depth z (this is the inverse of eq. (2)).
In order to know the temporal field received in the medium, an inverse temporal Fourier transform of the computed field A(x,z,ω) is performed.

3. Time-Domain Beamforming

Another possibility is to work everything in time domain. If s(i,t) is the temporal trace field received by transducer element i from the contralateral transducer, the geometrical delays having been removed for aligning the signals (these signals are affected by both amplitude and phase aberrations as well as waveform distortion). τ(i) are the delays applied to all transducer elements to achieve transmit focusing, i.e. in order to focus on point depth z₀, azimuth x₀ one has

$$τ ( i ) = \frac{1}{c} \sqrt{{( x ( i ) - {x}_{0} )}^{2} + {z}_{0}^{2}} .$$

The sent signals (through the aberrator) in time domain can thus be written as

sₛₑₙₜ(i,t)=s(i,t−τ(i))A_{Apod}(i) (1)

(remember that the phase and amplitude aberration is in s(t)). Then, the temporal signal received in the field at (x_{f},z_{f}) is the sum of the contributions of what comes from all transducer elements:

s_{received}(x_{f},z_{f})=Σᵢsₛₑₙₜ(i,t+τ(i,x_{f},z_{f})) (2)

With τ(i,x_{f},z_{f}) being the time needed for the sound to go from transducer element i to the field point at (x_{f},z_{f}):

$$τ ( i , {x}_{f} , {z}_{f} ) = \frac{1}{c} \sqrt{{( x ( i ) - {x}_{f} )}^{2} + {z}_{f}^{2}} \qquad ( 3 )$$

In summary, getting the field at any point from the measured aberration and the known, applied transmit wavefront, includes:
Measuring the temporal signals received by all transducer elements from the contralateral transducer to get s(i,t);
Applying the desired transmit beamforming parameters (apodization and time-delaying), as in eq. (1);
Simulating propagation to any field point by applying delays to the measured traces, as in eq. (3).

FIG. 10 exemplifies a transcranial imaging/therapy aberration prediction/correction process 1000. In an aberration estimation procedure, ultrasound 164 is transmitted through an inhomogeneous medium 168 and contralaterally received. The transmitting is done, to some degree sequentially, on a point source basis, and receiving is by means of a 2D transducer array 104, 108. Element-wise on the receiving array 104, 108, relative time delay and/or amplitude attenuation and/or distortion are estimated. The estimate, possibly in the form of the aberration maps 402, 404, 406, is used to select placement/extent of an acoustic window 204, 208, 212, the array 104, 108 on the side particular to the estimate being correspondingly translated if such is found to be appropriate. The procedure may be iterative, and repeated by again sending transmissive ultrasound, etc. (step S1004). The aberration estimation is repeated contralaterally, so that the temporal bone 172, 176 on the other side is accounted for in terms of aberration. This step may be intermixed with activity in the previous step, i.e., step S1004 (step S1008). Aberration maps 402, 404, 406 may be formed and, if so, are displayable on the apparatus display 148. As mentioned above in connection with step S1004, aberration maps 402, 404, 406 may already have been formed and utilized (step S1012). If beam shape is to be predicted (step S1016), it is done based on the transmit beamforming parameters and the aberration estimate, and the prediction 920 is available for display on the apparatus display 148 (step S1020). If, based interactively on the displayed prediction 920 of the aberrated beam 930, a setting of the device 110 is to be modified that would change the aberration estimate and/or the transmit beamforming parameters used in the beam shape prediction (step S1024), the modification is made (step S1028). Otherwise, if no such (further) modification is to be made or beam shape is not to be predicted, ultrasound correction, e.g., phase delay correction or patch contribution weighting for beamforming, is performed based on a result of the aberration estimation (step S1032). To predict the intensity of an ultrasound therapeutic beam, a contralateral arrangement of transducer arrays 104, 108 is provided (and typically both arrays would already have been provided at this point in the process 1000) (step S1036). Bubbles are supplied, e.g., intravenously, to a treatment or reference region 808, 816 (step S1040). Ultrasound intensity is monitored incrementally for the onset of subharmonic emission from contrast microbubbles within the treatment region 808, or from within a reference region 816 close to the treatment region (step S1044). The therapeutic beam 720, aberration-corrected by virtue of device setting modification is applied to the treatment region 730. Receive beamforming, from both sides of the skull 132, can draw on device modification previously performed based on respective aberration estimation results for the two sides (step S1048). The two acquired images are correlated and compounded, thereby enhancing visualization of beam placement (step S1052).

Ultrasound aberration, especially in transcranial imaging or therapy, is corrected by capturing the laterally two-dimensional nature of the aberration in the ultrasound being received, as by means of a two-dimensional receiving transducer array. In some embodiments, transmissive ultrasound is applied through the temporal window and is, for example, emitted from one or more real or virtual point sources at a time, each point source being a single transducer element or patch or the geometrical focus of a collection of elements or patches. A patch may serve, in one aspect, as a small focused transducer in the near field. A contralateral array is, in one version, comprised of the point sources. In some aspects, aberration maps structured, independent-variable-wise, to correspond to the array structure of the receiving transducer embody aberration estimates, the ultrasound device being configured for improving ultrasound operation by modifying device settings to improve the location of ultrasound reception/transmission or correct beamforming. Enhancements include beam placement visualization, and intensity and beam shape prediction.

It should be noted that the above-mentioned embodiments illustrate rather than limit the invention, and that those skilled in the art will be able to design many alternative embodiments without departing from the scope of the appended claims. For example, the bilateral receive beams that have been corrected for aberration can be maintained to monitor for change in brain structure, while the contralateral arrangement remains affixed to the patient's skull. In the claims, any reference signs placed between parentheses shall not be construed as limiting the claim. Use of the verb “to comprise” and its conjugations does not exclude the presence of elements or steps other than those stated in a claim. The article “a” or “an” preceding an element does not exclude the presence of a plurality of such elements. The invention may be implemented by means of hardware comprising several distinct elements, and by means of a suitably programmed computer having a computer readable medium. The mere fact that certain measures are recited in mutually different dependent claims does not indicate that a combination of these measures cannot be used to advantage.

1. A device comprising:
a two-dimensional transducer array configured for receiving transmissive ultrasound that has passed through an inhomogeneous medium, wherein the transmissive ultrasound comprises ultrasound emitted for reception in a direction of propagation; and
a processor configured (i) for performing aberration estimation on the received ultrasound, wherein performing occurs in two spatial dimensions of the array so that the aberration estimation accounts for aberration laterally in the two spatial dimensions, and (ii) for controlling an ultrasound operation of the device in response to a result of said aberration estimation to improve the ultrasound operation, wherein controlling comprises (a) phase aberration correction and (b) transmit/receive weighting of transducer elements/patches.

2. The device of claim 1, wherein the processor is further configured for modifying, based on said result of said aberration estimation, a setting of said device so as to at least one of a) improve a location of at least one of ultrasound transmission and ultrasound reception; and b) correct beamforming of ultrasound.

3. The device of claim 2, wherein the processor modifies the device setting based on at least one of (i) a selected placement of an acoustic window, and (ii) a selected extent of the acoustic window.

4. The device of claim 2, wherein said result comprises at least one aberration map for which both elevation and azimuth are independent variables, and wherein said modifying the device setting is based on one or more of said at least one aberration map.

5. The device of claim 1, wherein said result comprises a plurality of aberration maps, each map having a spatial independent variable, wherein at least two of (i) signal time delay, (ii) signal amplitude and (iii) signal distortion comprise dependent variables of respective ones of said maps.

6. The device of claim 1, wherein said result comprises at least one of (i) a signal amplitude map and (ii) a signal distortion map, and wherein the processor is further configured for utilizing at least one of said signal amplitude or signal distortion maps to regulate, as a weighting map, contribution of either (a) individual transducer elements or (b) individual patches to beamforming.

7. The device of claim 1, further comprising:
a contralateral transducer array, wherein the contralateral transducer array is configured for receive beamforming from both sides from a single ultrasound transmit pulse.

8. The device of claim 7, wherein the processor is further configured for compounding images acquired on both sides by said beamforming.

9. The device of claim 7, wherein said transmissive ultrasound eminates from said contralateral transducer array, and wherein said processor is further configured for controlling the ultrasound operation of the device such that said beamforming takes into account receive aberration correction respectively based both on (i) said aberration estimation on received transmissive ultrasound and (ii) aberration estimation on contralaterally received transmissive ultrasound.

10. The device of claim 1, further comprising:
a contralateral transducer array configured for emitting, from point sources distributed over the contralateral transducer array, said transmissive ultrasound, wherein a point source comprises a patch or transducer element of the contralateral transducer array, and wherein said processor is further configured for, based on the performed aberration estimation, selecting an acoustic window.

11. The device of claim 1, further comprising an array placement adjustor configured for translating at least one of the two-dimensional array, and a contralateral array, by less than a size of a patch of the array to be translated.

12. The device of claim 1, further comprising, for placement contralaterally to said transducer array, a source of said transmissive ultrasound.

13. The device of claim 12, wherein said source comprises a patch, whose input is initially beamformed separately, that, for said performing, serves as a point source with respect to said array.

14. The device of claim 12, wherein said source comprises a contralateral array, wherein said processor is further configured for focusing, from said contralateral array, a beam on an outer surface of a temporal bone, the focus serving, for said performing, as a point source with respect to said transducer array.

15. The device of claim 1, wherein the passing through being through a portion of said medium, and wherein improving said ultrasound operation comprises tailoring ultrasound to characteristics of said portion.

16. (canceled)

17. (canceled)

18. A method comprising:
receiving, via a two-dimensional transducer array, at any given moment, in more than one spatial dimension, transmissive ultrasound that has passed through an inhomogeneous medium, wherein the transmissive ultrasound comprises ultrasound emitted for reception in a direction of propagation from a contralateral two-dimensional transducer array; and
performing, via a on the received ultrasound, aberration estimation that correspondingly accounts for aberration laterally in said more than one spatial dimension, and controlling an ultrasound operation in response to a result of said aberration estimation to improve the ultrasound operation, wherein controlling comprises (a) phase aberration correction and (b) transmit/receive weighting of transducer elements/patches of a corresponding two-dimensional transducer array.

19. The method of claim 18, further comprising:
correcting aberration by modifying phase delays based on a phase delay map having said more than one spatial dimension, wherein correcting aberration comprises using relative time lags between respective pairs of elements of said aberration map in said modifying.

20. (canceled)

21. (canceled)

22. A non-transitory computer readable medium embodied with a computer program for enabling, through the use of a two-dimensional transducer array to receive transmissive ultrasound that has passed through an inhomogeneous medium, wherein the transmissive ultrasound comprises ultrasound emitted for reception in a direction of propagation, improvement of ultrasound operation, wherein said computer program includes instructions executable by a processor to perform a plurality of acts, said plurality comprising the act of:
performing aberration estimation on the received transmissive ultrasound, wherein performing occurs in two spatial dimensions of the array so that the aberration estimation accounts for aberration laterally in the two spatial dimensions, and
controlling the ultrasound operation in response to a result of said aberration estimation to achieve said improvement, wherein controlling comprises (a) phase aberration correction and (b) transmit/receive weighting of transducer elements/patches.

23. (canceled)
